(19) United States
(12) Patent Application Publication
(10) Pub. No.: US 2022/0233604 A1
(43) Pub. Date: Jul. 28, 2022

(54) METHODS AND COMPOSITIONS FOR TREATING CARDIOMYOCYTES

(71) Applicant: The Board of Trustees of the Leland Stanford Junior University, Stanford, CA (US)

(72) Inventors: Phillip C. Yang, Stanford, CA (US); Gentaro Ikeda, Los Altos, CA (US); Connor Galen O'Brien, Palo Alto, CA (US)

(21) Appl. No.: 17/611,272

(22) PCT Filed: May 14, 2020
(86) PCT No.: PCT/US20/32982

§ 371 Date: Nov. 15, 2021

Related U.S. Application Data
(60) Provisional application No. 62/848,486, filed on May 15, 2019.

(51) Int. Cl. A61K 35/34; C12N 5/077; A61K 9/127; A61P 9/00; A61P 39/00

(52) U.S. Cl. CPC A61K 35/34; C12N 5/0657; A61K 9/127; C12N 2501/415; A61P 39/00; C12N 2506/45; A61P 9/00

(57) ABSTRACT

Provided herein are compositions and methods for the restoration of intracellular bioenergy in disorders characterized by intracellular bioenergetics imbalance. Specifically, compositions and methods provided herein include extracellular vesicles that include mitochondria and/or mitochondrial fragments.

### CROSS-REFERENCED APPLICATIONS

This application claims priority to U.S. Provisional Application Ser. No. 62/858,486, filed May 15, 2019, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under contract HL130553 awarded by the National Institutes of Health. The Government has certain rights in the invention.

### BACKGROUND

Pharmacological therapies have improved survival of heart failure (HF) patients over the past three decades. Despite many pioneering medical therapies, HF is the leading cause of hospital admission in the US and 5-year survival is still a dismal 50%. HF represents bioenergetics imbalance. The disruption of the balance between energy supply and demand is important for the pathogenesis of HF. Cardiac tissue from patients with dilated, hypertrophic, and ischemic cardiomyopathy exhibits mitochondrial structural abnormalities and diminished ATP production, albeit with an increased metabolic energy demands in the failing heart. Insufficient energy generation results in loss of cardiomyocyte contractility, myocardial pump dysfunction, and ultimately decompensated HF. The current standard medical regimen include beta-blockade, diuresis, and renin-angiotensin-aldosterone antagonism. These pharmacologic agents attempt to correct this imbalance by reducing cardiac workload, namely, energy demand. These therapeutics are essentially non-curative because they do not target the primary energy source of the failing heart. In addition to reduced capacity to generate ATP, mitochondrial abnormality in HF are directly related to disease progression. Abnormal mitochondria become a major source of reactive oxygen species (ROS) and promote cell death through opening of mitochondrial permeability transition pore. Mitochondrial quality is maintained through mitochondrial biogenesis, mitochondrial dynamics, and mitochondrial autophagy (mitophagy). Peroxisome proliferator-activated receptor-y; coactivator-1 alpha (PGC-1α) serves as a master regulator of mitochondrial biogenesis. It is reported that PGC-1α expression levels are decreased in the myocardium from patients with HF. Transplantation of isolated mitochondria extracted from homogenized cells have been demonstrated to reduce infarct size in murine models of myocardial injury (MI). More recently, this form of mitochondrial transfer has been performed in a neonate with hypoplastic heart. However, the beneficial effects of this therapeutic approach are reported to be of limited benefit due to the following two reasons; (1) isolated mitochondria are critically damaged in extracellular environment with high calcium concentrations and (2) isolated mitochondria rarely pass through the cellular membrane.

### BRIEF SUMMARY

In view of the foregoing, there is a need for innovative therapy of cardiac disorders that targets the intracellular bioenergetics directly. The present disclosure addresses this need, and provides additional benefits as well.

In an aspect, provided herein are methods of treating a subject having a disorder characterized by intracellular bioenergetics imbalance in a cardiomyocyte. The methods include administering a therapeutically effective amount of cardiomyocyte mitochondria-containing extracellular vesicles.

In an aspect, provided herein are methods of treating a subject having a disorder characterized by intracellular bioenergetics imbalance in a cardiomyocyte. The methods include obtaining a sample of blood, isolating peripheral blood mononucleocyte cells (PBMCs) from the blood to obtain a PBMC fraction of PBMCs, inducing pluripotency of said PBMC fraction of PBMC's thereby producing induced pluripotent stem (iPS) cells, differentiating the iPS cells thereby producing cardiomyocytes, isolating cardiomyocyte mitochondria-containing extracellular vesicles produced from said cardiomyocytes; and administering a therapeutically effective amount of said cardiomyocyte mitochondria-containing extracellular vesicles to the subject.

In an aspect, provided herein are methods for improving cellular function in a cardiomyocyte including contacting the cardiomyocyte with cardiomyocyte mitochondria-containing extracellular vesicles.

In an aspect, provided herein are methods for improving the bioenergetics balance of a cardiomyocyte including contacting the cardiomyocyte with cardiomyocyte mitochondria-containing extracellular vesicles.

In an aspect, provided herein are methods for improving viability of a cardiomyocyte including contacting the cardiomyocyte with cardiomyocyte mitochondria-containing extracellular vesicles.

In an aspect, provided herein are compositions including iPSC-derived cardiomyocytes, cell culture media, and extracellular vesicles, where the extracellular vesicles are greater than 200 nm and include full mitochondria and/or mitochondrial fragments.

### BRIEF DESCRIPTION OF THE DRAWINGS

FIG. 1 shows a schematic representation of next generation therapy for heart failure, extracellular vesicles-mediated mitochondrial transplantation.

FIGS. 2A-2F show characteristics of mitochondria-rich extracellular vesicles (Mito-EVs). FIG. 2A shows that flow cytometry (FCM) revealed that the extracellular vesicles (EVs) in induced-cell culture medium (iCM) contained functional mitochondria. FIG. 2B shows a method established to collect mitochondria-rich EVs (Mito-EVs) using differential centrifugation. This method resulted in significantly increased mitochondria+-EVs and approximately 3 times more mitochondrial mass in EVs. FIG. 2C shows FCM plots of Mito-EVs. FIG. 2D is a bar graph showing the ratio of mitochondrial mass. FIG. 2E is a graph showing nanoparticle tracking analysis data. FIG. 2F is a graph showing the percentage of EVs positive for Mitotracker Green, for different size ranges (>180 nm, 180-300 nm, and 300-1300 nm). Size distribution was examined by Nanoparticle Tracking Analysis and FCM.

FIGS. 3A-3D show that Mito-EVs restored bioenergetics in hypoxic iCMs. Mito-EVs restored the ATP level in the iCMs exposed to hypoxia (1% 02, 24 h). 200 nm filtration compromised therapeutic effects, suggesting that mitochondria+-EVs were indispensable. Mitochondria were isolated from homogenized iCMs, which contain 100 times more mitochondrial protein than Mito-EVs (3×10⁸/mL) but had no effect on the recovery of intracellular energetics. FIG. 3A shows bar graphs presenting intracellular ATP 3 hours after reoxygenation for Mito-EVs or Mito-EVs+200 nm filter (Md-EVs) at different concentrations and in condition of hypoxia or not. FIG. 3B is a picture of a Western-blot showing immunostaining for VDAC or COX4 in Mito-EVs (30×107 Mito-EVs per mL) or in isolated mitochondria protein (0.03, 0.1, 0.3 and 1 μg/mL). FIG. 3C shows bar graphs presenting intracellular ATP 24 hours after reoxygenation for Mito-EVs or Mito-EVs+200 nm filter (Md-EVs) at different concentrations and in condition of hypoxia or not. FIG. 3D shows bar graphs presenting intracellular ATP in isolated mitochondria in condition of hypoxia or not.

FIG. 4A-4F show that Mito-EVs increased PGC-la protein expression in hypoxic iCM and improved mitochondrial quality. Mito-EV from iCMs expressed PGC-1α mRNA, a key regulator of mitochondrial biogenesis, 64-fold greater than Mito-EVs from iPSC. Mito-EVs treatment increased PGC-1α protein level in the recipient iCMs. They improved mitochondrial quality (mitochondrial membrane potential and mitochondrial ROS) at 24 hours after treatment. FIG. 4A is a bar graph showing PGC-1α mRNA in EVs. FIG. 4B is a Western-blot showing immunostaining of PGC-1α, COX IV and β-actin in Mito-EVs, Md-EVs, in comparison to a control experiment. FIG. 4C is a bar graph showing PGC-1α mRNA in Mito-EVs, and-EVs, and in a control experiment. FIG. 4D show bar graphs showing mDNA to nDNA ratios in Mito-EVs, Md-EVs, and in a control experiment, 3 or 24 hours after reoxygenation. FIG. 4E is a bar graph showing membrane potential ratios for Mito-EVs, 24 hours after reoxygenation, in comparison to a control experiment. FIG. 4F is a bar graph showing the percentage mitrochondrial reactive oxygen species (ROS) in Mito-EVs, Md-EVs, in comparison to a control experiment, 24 hours after reoxygenation.

FIGS. 5A-5D show that intramyocardial injection of Mito-EVs (3.0×10⁸) improved LV remodeling at 4 weeks as measured by MRI. In a murine model of myocardial infarction, intramyocardial injection of Mito-EVs (3.0×10⁸) improved left ventricular ejection fraction (LVEF), end-diastolic volume (LVEDV), and end-systolic volume (LVESV) significantly at 4 weeks as measured by MRI. FIG. 5A MRI shows pictures of a murine model of myocardial infarction after injection of Mito-EVs, Md-EVs or of a Control, in Diastole or Systole. FIG. 5B is a bar graph showing percentage of LVEF. FIG. 5C is a bar graph showing LVDD in μL. FIG. 5D is a bar graph showing LVDS in μL.

FIGS. 6A-6E show that Mito-EVs increased myocardial viability in vivo mouse model of myocardial injury, when treated with a control, Mito-EVs, or Md-EVs, at week 4 after treatment. FIG. 6A shows myocardial viability in the peri-infarct region as visualized by manganese-enhanced magnetic resonance imaging (MEMRI), highlighted with arrows. FIG. 6B is a bar graph showing viability percentage of viable myocardium. FIG. 6C is a bar graph showing left ventricle LV mass (mg). FIG. 6D is a bar graph showing lung/tibia ratios (mg/mm). FIG. 6E is a bar graph showing heart/tibia ratios (mg/mm). Overall, myocardial viability was significantly greater in Mito-EVs-treated animals at week 4 vs control. Mito-EVs significantly reduced lung weight at week 4.

FIGS. 7A-7B show that EVs-mediated transfer of functional mitochondria enhanced mitochondrial biogenesis and cellular energetics to restore the failing iCMs. FIG. 7A shows mitochondria positive-EVs in conditioned medium. FIG. 7B shows intracellular ATP production.

FIGS. 8A-8D show the pharmacodynamics of doxorubicin in induced cardiomyocytes (iCM). FIG. 8A shows viability data for MDiCM dose titration. Lines represent, from top to bottom: 0.1 μM, 1 μM, and 10 μM concentrations. FIG. 8B shows viability data for GLiCM dose titration. Lines represent, from top to bottom: 0.1 μM, 1 μM, and 10 μM concentrations. FIG. 8C shows viability data for SDiCM dose titration. Lines represent, from top to bottom: 0.1 μM, 1 μM, and 10 μM concentrations. FIG. 8D shows annexin-PI positivity data for SDiCM 1 μM doxorubicin exposure. After 96 hours, the lines represent, from top to bottom: early apoptosis, viability, late apoptosis, and necrosis.

FIGS. 9A-9D show that co-culture of MSCs improved cardiomyocyte viability and reduced apoptosis in a dose-dependent fashion. Paracrine mediators recapitulate this effect. FIG. 9A-9C show viability data for co-culture dose titration (FIG. 9A), exosome dose titration (FIG. 9B), and conditioned media dose titration (FIG. 9C). FIG. 9D shows annexin-PI positivity data for GLiCM annexin-PI. Bars represent, from left to right: viability, early apoptosis, late apoptosis, and necrosis.

FIGS. 10A-10B shows that conditioned media contains a unique population of microvesicles. Top graphs illustrate particle concentration for media containing exosomes less than 220 nm (FIG. 10A) and for conditioned media (FIG. 10B). Bottom graphs show FACS data with sizes depicted of greater than or less than 240 nm and greater than 500 nm, and quadrant data shown is as follows: Q1 1.32, Q2 2.09, Q3 96.6, and Q4 0.011 for the leftmost graph; and Q1 19.2, Q2 43.5, Q3 36.8, and Q4 0.53 for the rightmost graph.

FIGS. 11A-11B show that concentrated microvesicles demonstrated improved dose-response. FIG. 11A is a bar graph showing large microvesicle isolation results. FIG. 11B is a bar graph showing GLiCM annexin-PI data. Bars in the right panel represent, from left to right: viability, early apoptosis, late apoptosis, and necrosis.

### DETAILED DESCRIPTION

I. Definitions

As used herein, the terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. The disease may be a cardiac disease. The disease may be cardiomyopathy.

As used herein, the terms “heart failure” or “HF” also known as “chronic heart failure” or “CHF”, refer to when the heart is unable to pump sufficiently to maintain blood flow to meet the body's needs. Causes of heart failure include coronary artery disease including a previous myocardial infarction (heart attack), high blood pressure, atrial fibrillation, valvular heart disease, excess alcohol use, infection, and cardiomyopathy of an unknown cause. These cause heart failure by changing either the structure or the functioning of the heart. Heart failure may be caused by any condition that reduces the efficiency of the heart muscle through damage or overloading. As such, it can be caused by a variety of conditions, including myocardial infarction (in which the heart muscle is starved of oxygen and dies), hypertension (which increases the force of contraction needed to pump blood) and amyloidosis (in which misfolded proteins are deposited in the heart muscle, causing it to stiffen). Over time, these increases in workload will produce changes to the heart itself.

As used herein, the term “cardiomyopathy” refers to a group of diseases that negatively affect the heart muscle. Types of cardiomyopathy include hypertrophic cardiomyopathy, dilated cardiomyopathy, restrictive cardiomyopathy, arrhythmogenic right ventricular dysplasia, and takotsubo cardiomyopathy (broken heart syndrome). In hypertrophic cardiomyopathy, the heart muscle enlarges and thickens. In dilated cardiomyopathy, the ventricles enlarge and weaken. In restrictive cardiomyopathy, the ventricle stiffens. Ischemic cardiomyopathy is a type of cardiomyopathy caused by a narrowing of the coronary arteries that supply blood to the heart.

As used herein, the terms “treating”, or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination. The term “treating” and conjugations thereof may include prevention of an injury, pathology, condition, or disease. In embodiments, treating is preventing. In embodiments, treating does not include preventing.

As used herein, the terms “treating” and “treatment” may include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

As used herein, the terms “patient” or “subject in need thereof” refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term an “effective amount” refers to an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” is an amount that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations.

As used herein, the term “therapeutically effective amount” refers to that amount of the therapeutic agent sufficient to ameliorate the disorder, as described above. Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

As used herein, the term “administering” includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intramyocardial, intracoronary, intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In embodiments, the administering does include administration of any active agent other than the recited active agent.

As used herein, the term “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair.

As used herein, the terms “induced pluripotent stem cell” or “iPS cell” or “iPSC” refer to a type of pluripotent stem cell that can be generated directly from adult cells. The generation of iPS cells is c dependent on the transcription factors used for the induction. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability.

As used herein, the terms “peripheral blood mononuclear cell” or “PBMC” refers to any peripheral blood cell having a single, round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. In humans, lymphocytes make up the majority of the PBMC population, followed by monocytes, and only a small percentage of dendritic cells. These cells can be extracted from whole blood using Ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes.

As used herein, the terms “cardiomyocyte” and “cardiac muscle cells” also known as “cardiac myocytes” refer to the muscle cells within the cardiac muscle (heart muscle). Each myocardial cell contains myofibrils, which are specialized organelles consisting of long chains of sarcomeres, the fundamental contractile units of muscle cells. Cardiomyocytes show striations similar to those on skeletal muscle cells. Unlike multinucleated skeletal cells, the majority of cardiomyocytes contain only one nucleus, although they may have as many as four. Cardiomyocytes have a high mitochondrial density, which allows them to produce adenosine triphosphate (ATP) quickly, making them highly resistant to fatigue.

As used herein, the term “intracellular bioenergy” refers to chemical potential energy, such as ATP, generated and stored in cells for the purposes of executing cell functions and metabolism. The energy can be produced by both aerobic and anaerobic mechanisms. As used herein, the term “intracellular bioenergetics imbalance” refers to a deficit in the production or storage of energy, such as ATP, such that there is insufficient energy supply to meet intracellular needs. This can result in low performance of the cell and/or disease. A number of diseases or conditions may result from, be caused by, or result in intracellular energetic imbalance, for example, cardiomyopathy.

As used herein, the term “mitochondrion” (plural mitochondria) refers to a double-membrane-bound organelle found in most eukaryotic organisms. Some cells in some multicellular organisms may lack them (for example, mature mammalian red blood cells). Mitochondria are commonly between 0.75 and 3 μm in diameter but vary in size and structure. The most prominent role of mitochondria is to produce the energy currency of the cell, ATP (i.e., phosphorylation of ADP), through respiration, and to regulate cellular metabolism.

As used herein, the term “extracellular vesicle” refers to a nanosized, membrane-bound vesicle released from cells that can transport cargo, including DNA, RNA, and proteins, between cells as a form of intercellular communication. Different EV types, including microvesicles (MVs), exosomes, oncosomes, and apoptotic bodies, have been characterized based on their biogenesis or release pathways. The content of EVs includes lipids, nucleic acids, proteins, and organelles from donor cells. EVs are typically isolated by the following methods: differential centrifugation culminating in ultrafiltration, density gradient/cushion centrifugation, and immunoaffinity-based capture.

As used herein, the term “microvesicle” refers to a subtype of extracellular vesicles that bud directly from the plasma membrane, are typically 100 nanometers (nm) to 1 micrometer (μm) in size, and contain cytoplasmic cargo.

As used herein, the term “exosome” refers to a subtype of extracellular vesicles that is formed by the fusion between multivesicular bodies and the plasma membrane, in which multivesicular bodies release smaller vesicles (exosomes) whose diameters typically range, but not exclusively, from 40 to 120 nm.

As used herein, the term “cardiomyocyte mitochondria-containing extracellular vesicles” refers to extracellular vesicles derived from cardiomyocytes that contain full (whole) mitochondria or mitochondrial fragments. Mitochondria-containing extracellular vesicles may be generated from either 1) specifically allogeneic human mesenchymal stem cells and autologous cardiomyocytes or 2) human cells. In embodiments, where the extracellular vesicles derived from cardiomyocytes contain mitochondrial fragments, the mitochondrial fragments are at least partially capable of performing the biological function of a whole mitochondria (e.g. produce ATP and/or to regulate cellular metabolism).

As used herein, the term “hypoxia” is a condition in which the body or a region of the body is deprived of adequate oxygen supply at the tissue or cellular level.

As used herein, the term “chemotherapeutic agents” or “anti-cancer drugs” refer to a type of treatment for cancer. Traditional chemotherapeutic agents are cytotoxic by means of interfering with cell division (mitosis) but cancer cells vary widely in their susceptibility to these agents. Chemotherapeutic agents damage or stress cells, which may then lead to cell death if apoptosis is initiated.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity or characteristic of a cell in the absence of treatment as described herein (including embodiments and examples).

As used herein, the term “mitochondria]. reactive oxygen species” or “mitochondrial ROS” or “mtROS” or “mROS” are reactive oxygen species (ROS) that are produced by mitochondria. Generation of mitochondrial ROS mainly takes place at the electron transport chain located on the inner mitochondrial membrane during the process of oxidative phosphorylation (OXPHOS). Leakage of electrons at complex I and complex III from electron transport chains leads to partial reduction of oxygen to form superoxide. Subsequently, superoxide is quickly dismutated to hydrogen peroxide by two dismutases including superoxide dismutase 2 (SOD2) in mitochondria′ matrix and superoxide dismutase 1 (SOD′) in mitochondrial intermembrane space. Collectively, both superoxide and hydrogen peroxide generated in this process are considered as mitochondrial ROS. Mitochondria! ROS are viewed as important signaling molecules, whose levels of generation can vary depending on cellular energy supply and demand. At low levels, mitochondria′ ROS are may be important for metabolic adaptation as seen in hypoxia. Mitochondrial ROS, stimulated by danger signals such as lysophosphatidylcholine, and Toll-like receptor 4 and Toll-like receptor 2 bacterial ligands lipopolysaccharide (LPS) and lipopeptides, are involved in regulating inflammatory response. Finally, high levels of mitochondria″ ROS activate apoptosis/autophagy pathways capable of inducing cell death.

As used herein, the term “CHIR” refers to a kinase inhibitor of GSK3α and GSK3β, with off-target effects on kinases within the CDK2-cyclin A2/E cell-cycle complex. CHIR can be used to induce cardiac differentiation of induced pluripotent stem cells. (See for example, Laco, F. et al. Stem Cell Reports. 2018 Jun. 5; 10(6): 1851-1866. Published online 2018 Apr. 26. doi:10.1016/j.stemcr.2018.03.023).

II. Compositions

In an aspect, provided herein are compositions including iPSC-derived cardiomyocytes, cell culture media, and extracellular vesicles, where the extracellular vesicles are greater than 200 nm and include mitochondria and/or mitochondrial fragments.

In embodiments, the cell culture media includes E8 medium for iPSC culture and RPMI media (+glutamax) with B27 supplement by Thermo Fisher for the iCMs. In embodiments, the cell culture media include Dulbecco's high glucose media with 10% fetal bovine serum for the mesenchymal stem cells.

In embodiments, the extracellular vesicles are between about 200 nm and 1000 nm. In embodiments, the extracellular vesicles are about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm. In embodiments, the extracellular vesicles are about 250 nm. In embodiments, the extracellular vesicles are about 300 nm. In embodiments, the extracellular vesicles are about 350 nm. In embodiments, the extracellular vesicles are about 400 nm. In embodiments, the extracellular vesicles are about 450 nm. In embodiments, the extracellular vesicles are about 500 nm. In embodiments, the extracellular vesicles are about 550 nm. In embodiments, the extracellular vesicles are about 600 nm. In embodiments, the extracellular vesicles are about 650 nm. In embodiments, the extracellular vesicles are about 700 nm. In embodiments, the extracellular vesicles are about 750 nm. In embodiments, the extracellular vesicles are about 800 nm. In embodiments, the extracellular vesicles are about 850 nm. In embodiments, the extracellular vesicles are about 900 nm. In embodiments, the extracellular vesicles are about 950 nm. In embodiments, the extracellular vesicles are about 1000 nm.

In embodiments, mitochondrial fragments include presence of mitochondria that may initially confirmed by staining concentrated supernatant from cell culture with mitotracker deep red and mitotracker green. Mitotracker green demonstrates the presence of mitochondria while mitotracker deep red is concentrated at mitochondrial membranes actively holding electrical potential in the electron transport chain, a marker of mitochondrial viability. These protein composition of these particles then confirmed by western gel, which has demonstrated substantial quantities of proteins specific to mitochondrial membranes, VDAC and COX4. These findings confirm that the membrane is active and composed of proteins specific to mitochondria. The size of these fragments cannot be accurately discerned by electron microscopy, so no formal definition of a mitochondrial fragment can be firmly applied.

In embodiments, the extracellular vesicles have the ability to restore ATP levels in a cell that has reduced ATP compared to a control cell. In embodiments, the control cell is a cell that has not been treated with mitochondria containing vesicles. In embodiments, the control cell is a non-injured cardiomyocyte.

In embodiments, the extracellular vesicles have the ability to improve mitochondrial membrane potential in a cell that has reduced membrane potential compared to a control cell. In embodiments, the control cell is a cell that has not been treated with mitochondria containing vesicles. In embodiments, the control cell is a non-injured cardiomyocyte.

In embodiments, the extracellular vesicles have the ability to reduce the percentage of mitochondrial reactive oxygen species in a cell that has a higher percentage of mitochondrial reactive oxygen species compared to a control cell. In embodiments, the control cell is a cell that has not been treated with mitochondria containing vesicles. In embodiments, the control cell is a non-injured cardiomyocyte.

In embodiments, the extracellular vesicles express PGC-1α. In embodiments, the extracellular vesicles induce expression of PGC-1α in a recipient cell.

III. Methods of Use

In an aspect, provided herein are methods of treating a subject having a disorder characterized by intracellular bioenergetics imbalance in a cardiomyocyte. The methods include administering a therapeutically effective amount of cardiomyocyte mitochondria-containing extracellular vesicles.

In an aspect, provided herein are methods of treating a subject having a disorder characterized by intracellular bioenergetics imbalance in a cardiomyocyte. The methods include obtaining a sample of blood, isolating peripheral blood mononucleocyte cells (PBMCs) from the blood to obtain a PBMC fraction of PBMC's, inducing pluripotency of said PBMC fraction of PBMC's thereby producing induced pluripotent stem (iPS) cells, differentiating the iPS cells thereby producing cardiomyocytes, isolating cardiomyocyte mitochondria-containing extracellular vesicles produced from said cardiomyocytes; and administering a therapeutically effective amount of said cardiomyocyte mitochondria-containing extracellular vesicles to the subject.

In embodiments, disorders characterized by intracellular bioenergetics imbalance in a cardiomyocyte include heart failure and cardiomyopathy. In embodiments, cardiomyopathy includes hypertrophic cardiomyopathy, dilated cardiomyopathy, restrictive cardiomyopathy, arrhythmogenic right ventricular dysplasia, and ischemic cardiomyopathy. In embodiments, the disorder is heart failure. In embodiments, the disorder is hypertrophic cardiomyopathy. In embodiments, the disorder is dilated cardiomyopathy. In embodiments, the disorder is restrictive cardiomyopathy. In embodiments, the disorder is arrhythmogenic right ventricular dysplasia. In embodiments, the disorder is ischemic cardiomyopathy.

In embodiments, the methods include isolating peripheral blood mononucleocyte cells (PBMCs) from a sample of blood to obtain a PBMC fraction of PBMCs. In embodiments, isolating peripheral blood mononucleocyte cells is accomplished by any method known to isolate PBMC for example, density centrifugation, utilization of cell preparation tubes (CPTs), and utilization of SepMate tubes with freshly collected blood. In embodiments, isolating PBMCs is achieved by density centrifugation. In embodiments, density centrifugation includes Ficoll or Ficoll-Pague as a density gradient medium.

In embodiments, the methods include inducing pluripotency of peripheral blood mononucleocyte cells thereby producing induced pluripotent stem cells. In embodiments, inducing pluripotency may be accomplished by any method know to induce pluripotency including for example by exposing cells to factors that induce pluripotency (See, for example, Takahashi K. Yamanaka S (August 2006). “Induction of pluripotent stein cells from mouse embryonic and adult fibroblast cultures by defined factors”. Cell. 126 (4): 663-76). In embodiments, factors that induce pluripotency include for example Klf4-Oct3/4-Sox2 (KOS) and L-Myc. In embodiments, inducing pluripotency of a PBMC fraction of PBMC's thereby producing induced pluripotent stem (iPS) cells is accomplished by exposing the PBMCs to one or more factors selected from Klf4-Oct3/4-Sox2 (KOS) and L-Myc.

In embodiments, the methods include differentiating induced pluripotent stem cells thereby producing cardiomyocytes. Methods of differentiating induced pluripotent stem cells into cardiomyocytes include the use of certain media and/or supplements or combinations thereof. In embodiments, the combination of media and supplements includes one or more selected from RPMI 1640, B27, rH-albumin, L-asorbic acid 2-phosphate, lactate, CH1R99021, and Wnt-059. In embodiments, methods for differentiating induced pluripotent stem cells include treatment with 6 μM CHIR for 2 or 3 days followed by treatment with C59 for 2 days. During differentiation, cells were cultured with RPMI medium with B27 supplement without insulin.

In embodiments, the methods include isolating extracellular vesicles that include mitochondria and/or mitochondrial fragments. In embodiments, the extracellular vesicles that include mitochondria and/or mitochondrial fragments are isolated from induced pluripotent stem cells (iPSC), induced pluripotent stem cell derivatives, induced cardiomyocytes (iCMs) or conditioned medium from any of the aforementioned. In embodiments, conditioned media is spent media harvested from cultured cells. It contains metabolites, growth factors, and extracellular matrix proteins secreted into the medium by the cultured cells. In embodiments, the cultured medium may be from induced pluripotent stem cells (iPSC). In embodiments, the cultured medium may be from induced pluripotent stem cell derivatives. In embodiments, the cultured medium may be from induced cardiomyocytes (iCMs).

In embodiments, the extracellular vesicles that include mitochondria and/or mitochondrial fragments are isolated from induced pluripotent stem cells (iPSC). In embodiments, the extracellular vesicles that include mitochondria and/or mitochondrial fragments are isolated from induced pluripotent stem cell derivatives. In embodiments, the extracellular vesicles that include mitochondria and/or mitochondrial fragments are isolated from induced cardiomyocytes (iCM). In embodiments, the extracellular vesicles that include mitochondria and/or mitochondrial fragments are isolated from conditioned media from induced pluripotent stem cells (iPSC). In embodiments, the extracellular vesicles that include mitochondria and/or mitochondrial fragments are isolated from conditioned media from induced cardiomyocytes (iCM).

In embodiments, the methods include administering a therapeutically effective amount of mitochondrial extracellular vesicles to a subject. In embodiments, the methods include administering a therapeutically effective amount of cardiomyocyte mitochondria-containing extracellular vesicles. In embodiments, the methods include administering a therapeutically effective amount of iPSC mitochondrial extracellular vesicles. In embodiments, the methods include administering a therapeutically effective amount of iPSC-derivative mitochondrial extracellular vesicles.

In embodiments, the extracellular vesicles are between about 200 nm and 1000 nm. In embodiments, the extracellular vesicles are about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm. In embodiments, the extracellular vesicles are about 250 nm. In embodiments, the extracellular vesicles are about 300 nm. In embodiments, the extracellular vesicles are about 350 nm. In embodiments, the extracellular vesicles are about 400 nm. In embodiments, the extracellular vesicles are about 450 nm. In embodiments, the extracellular vesicles are about 500 nm. In embodiments, the extracellular vesicles are about 550 nm. In embodiments, the extracellular vesicles are about 600 nm. In embodiments, the extracellular vesicles are about 650 nm. In embodiments, the extracellular vesicles are about 700 nm. In embodiments, the extracellular vesicles are about 750 nm. In embodiments, the extracellular vesicles are about 800 nm. In embodiments, the extracellular vesicles are about 850 nm. In embodiments, the extracellular vesicles are about 900 nm. In embodiments, the extracellular vesicles are about 950 nm. In embodiments, the extracellular vesicles are about 1000 nm.

In embodiments, the extracellular vesicles that include mitochondria and/or mitochondrial fragments are autologous to the subject. In embodiments, the extracellular vesicles that include mitochondria and/or mitochondrial fragments are heterologous to the subject. In embodiments, the cardiomyocyte extracellular vesicles that include mitochondria and/or mitochondrial fragments are autologous to the subject. In embodiments, the cardiomyocyte extracellular vesicles that include mitochondria and/or mitochondrial fragments are heterologous to the subject. In embodiments, the induced pluripotent stem cell extracellular vesicles that include mitochondria and/or mitochondrial fragments are autologous to the subject. In embodiments, the induced pluripotent stem cell extracellular vesicles that include mitochondria and/or mitochondrial fragments are heterologous to the subject.

In an aspect, provided herein are methods for improving cellular function in a cardiomyocyte including contacting the cardiomyocyte with cardiomyocyte mitochondria-containing extracellular vesicles. In embodiments, improving cellular function is relative to absence of contacting the cardiomyocyte with cardiomyocyte mitochondria-containing extracellular vesicles. In embodiments, improving cellular function is relative to a standard control population of cardiomyocytes. In embodiments, the control population includes untreated damaged cardiomyocytes. In embodiments, improving cellular function is relative to non-diseased cardiomyocytes.

In an aspect, provided herein are methods for improving the bioenergetics balance of a cardiomyocyte including contacting the cardiomyocyte with cardiomyocyte mitochondria-containing extracellular vesicles. In embodiments, improving bioenergetics balance is relative to absence of contacting the cardiomyocyte with cardiomyocyte mitochondria-containing extracellular vesicles. In embodiments, improving bioenergetics balance is relative to a standard control population of cardiomyocytes. In embodiments, the control population includes untreated damaged cardiomyocytes. In embodiments, improving bioenergetics balance is relative to non-diseased cardiomyocytes.

In an aspect, provided herein are methods for improving viability of a cardiomyocyte including contacting the cardiomyocyte with cardiomyocyte mitochondria-containing extracellular vesicles. In embodiments, improving viability is relative to absence of contacting the cardiomyocyte with cardiomyocyte mitochondria-containing extracellular vesicles. In embodiments, improving viability is relative to a standard control population of cardiomyocytes. In embodiments, the control population includes untreated damaged cardiomyocytes. In embodiments, improving viability is relative to non-diseased cardiomyocytes.

In embodiments, the cardiomyocyte is an injured cell. In embodiments, the injured cell is an ischemic cell, an oxidative stressed cell, or a starved cell. In embodiments, the injured cell is an ischemic cell. In embodiments, the injured cell is an oxidative stressed cell. In embodiments, the injured cell is a starved cell. In embodiments, the injured cell is a cell exposed to one or more chemotherapeutic agents. In embodiments, the injured cell is injured due to ischemic cardiomyopathy. In embodiments, the injured cell is injured due to anthracycline induced cardiomyopathy. In embodiments, the injured cell is injured due to dilated cardiomyopathy. In embodiments, the injured cell is a cell exposed to hydrogen peroxide.

In embodiments, improved cellular function is selected from restored bioenergetics, increased intracellular ATP levels, and/or increased expression of PGC-1α. In embodiments, improved cellular function is selected from restored bioenergetics. In embodiments, improved cellular function is selected from increased intracellular ATP levels. In embodiments, improved cellular function is selected from increased expression of PGC-1α.

In embodiments, restored bioenergetics is measured as an increase in intracellular ATP levels in M-EV treated injured cells compared to the levels in control cells. In embodiments, the control cell is an untreated injured iCMs.

In embodiments, improved viability is measured as a decrease in the number or percentage of dead cells in a cardiomyocyte population compared to a control population. In embodiments, the control population includes untreated damaged cardiomyocytes. In embodiments, improving viability is relative to non-diseased cardiomyocytes. In embodiments, improving viability is relative to healthy, undamaged cardiomyocytes.

In embodiments, in vitro measurements of improved cellular function include improved iCM contractility, normalized iCM action potential duration and calcium transients. In embodiments, in vivo measurements of improved cellular function include improved LVEF, volume, viability, reduced scar formation/fibrosis, and apoptosis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

### Mitochondria-Containing Vesicles from iCM Contained Functional Mitochondria

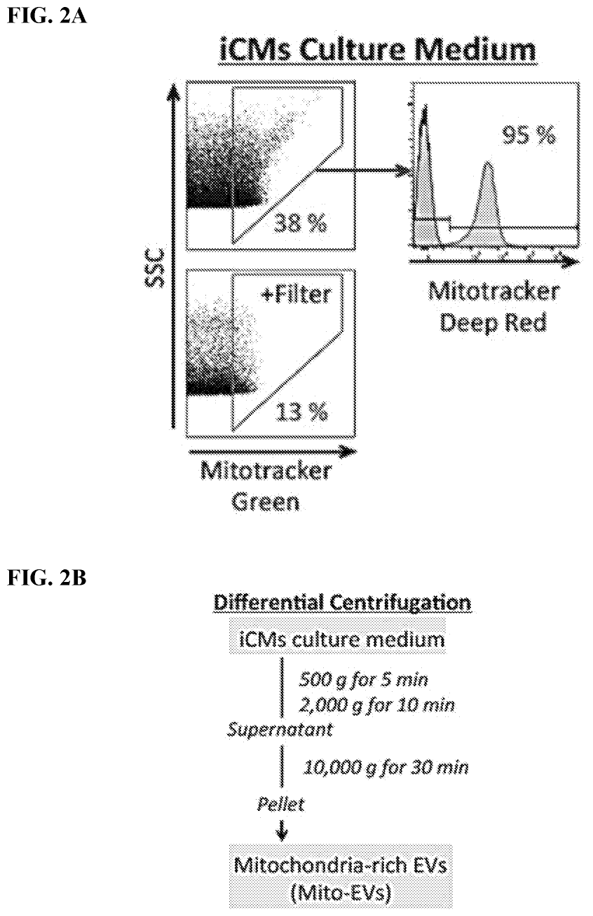

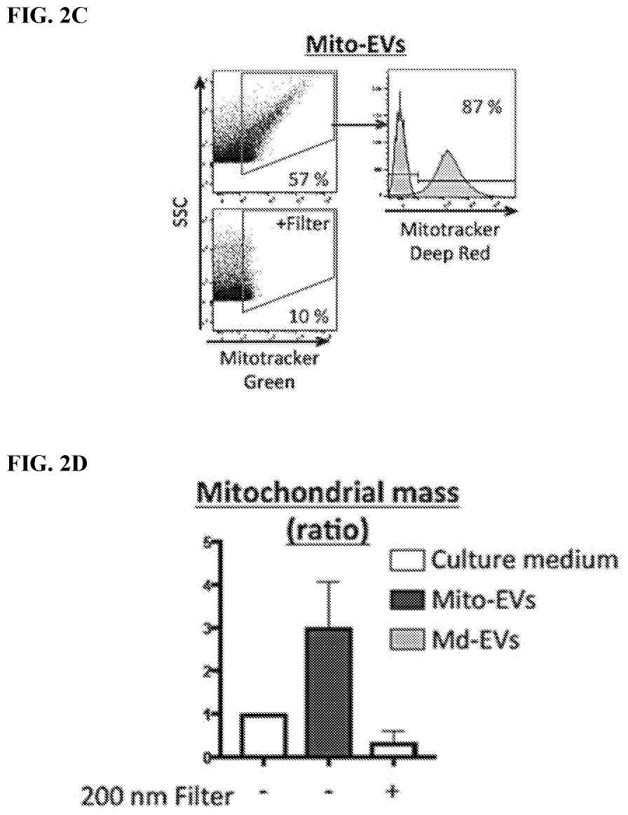

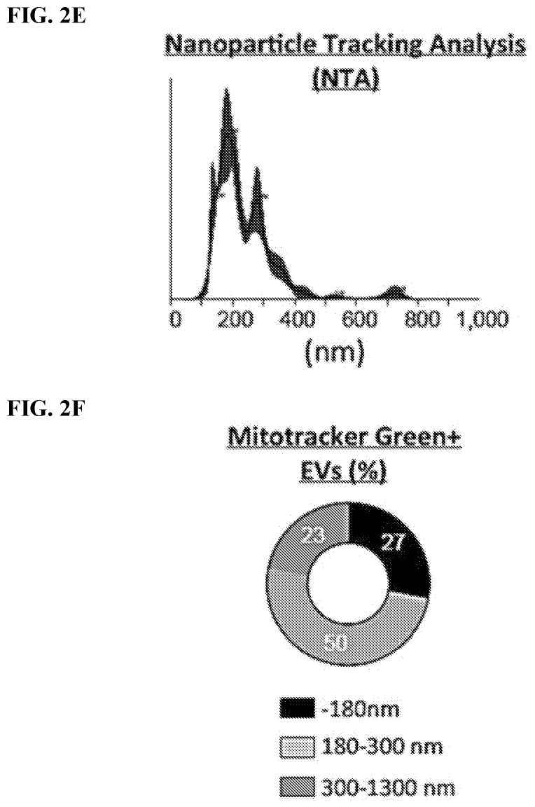

Mitochondria-containing extracellular vesicles (M-EVs) were collected by differential centrifugation (10,000 g for 30 min). Western blot revealed that M-EVs incorporated all electron transport chain (ETC) proteins. Transmission electron microscopy (TEM) revealed that mitochondria exist inside M-EVs. Flow cytometry (FCM) analysis revealed that M-EVs contained membrane potential positive mitochondria. M-EVs produce ATP. Nanosight and FCM revealed that the size of M-EVs ranged mostly 200-500 nm. MV also contained PGC-1α mRNA and protein. (FIGS. 2A-2F).

### Example 2

### Mitochondria-Containing Extracellular Vesicles Restore the Cellular Bioenergetics in iPSC Derived Human Cardiomyocytes After Hypoxic Injury

Experiments described herein demonstrated that human cardiomyocyte exosome cargo, including RNAs and proteins, were efficiently transferred to the injured human cardiomyocytes. The data demonstrated that the larger-size extracellular vesicles (EVs) contain functional mitochondria. They restored the injured myocardium leading to EV-mediated mitochondrial transfer.

The goal of this study was to achieve pre-clinical proof of concept for the efficacy of EVs-mediated mitochondrial transfer. The hypothesis tested was that EVs-mediated mitochondrial transfer restores the failing heart by reactivating the intracellular bioenergetics.

Materials and Methods

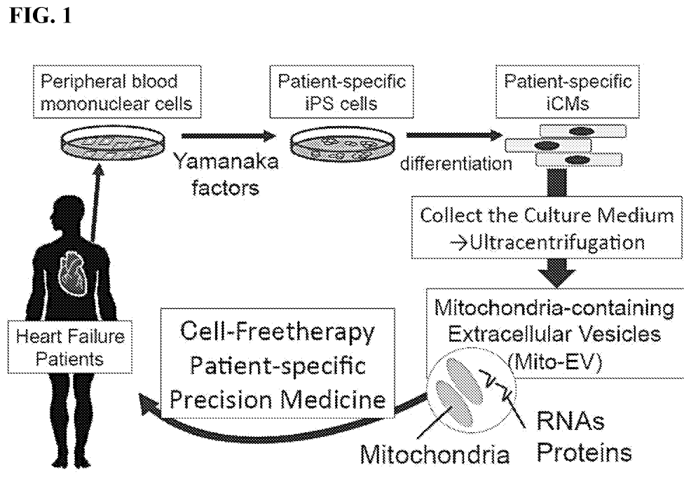

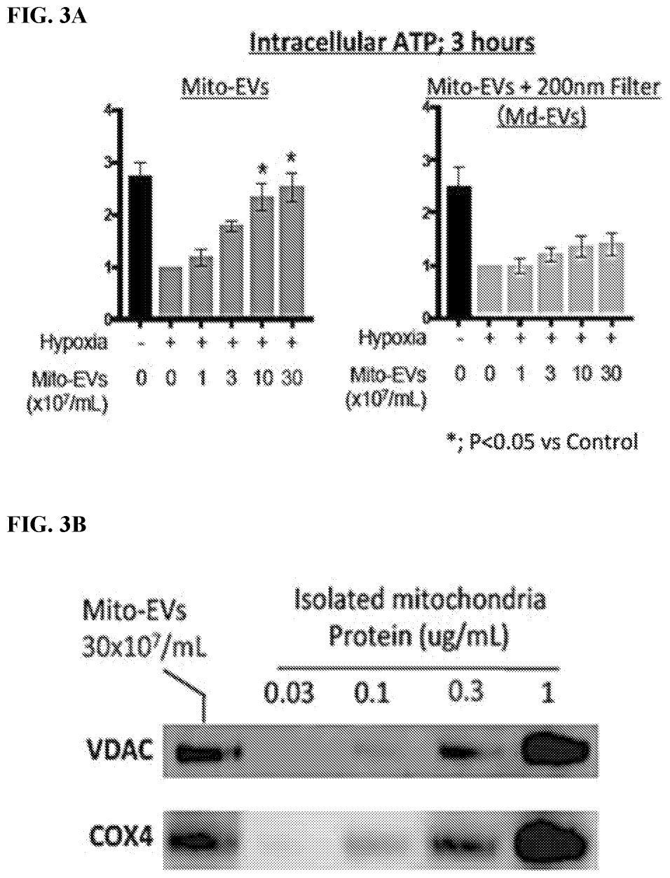

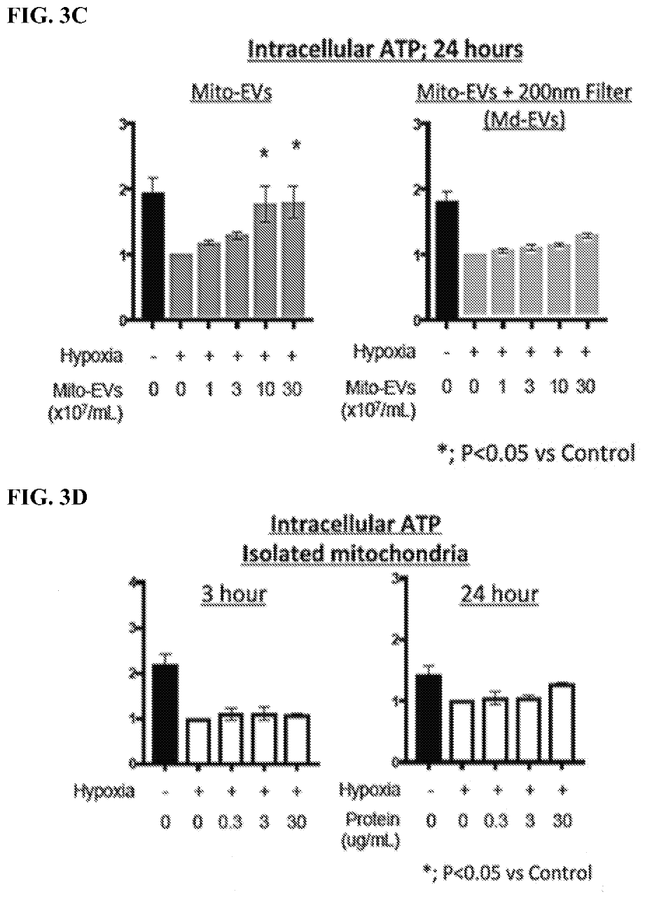

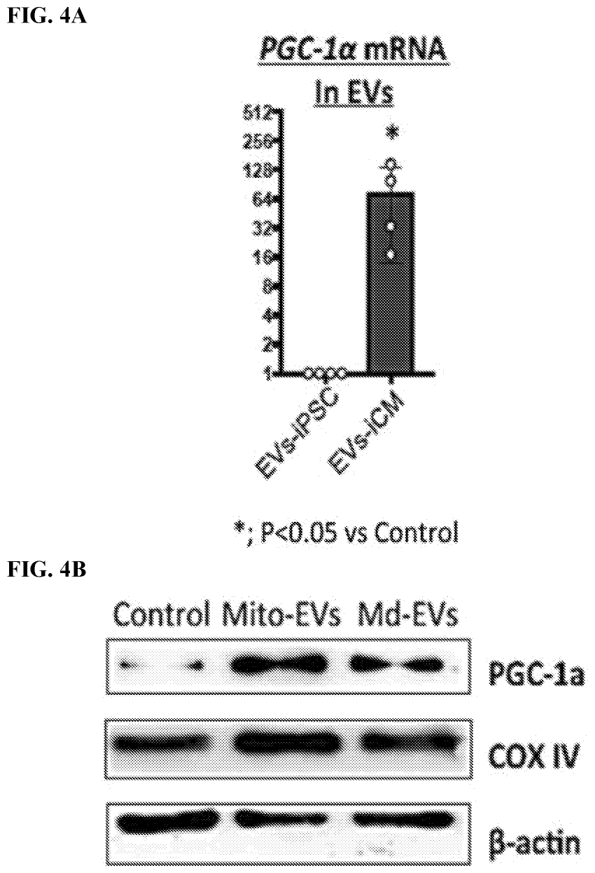

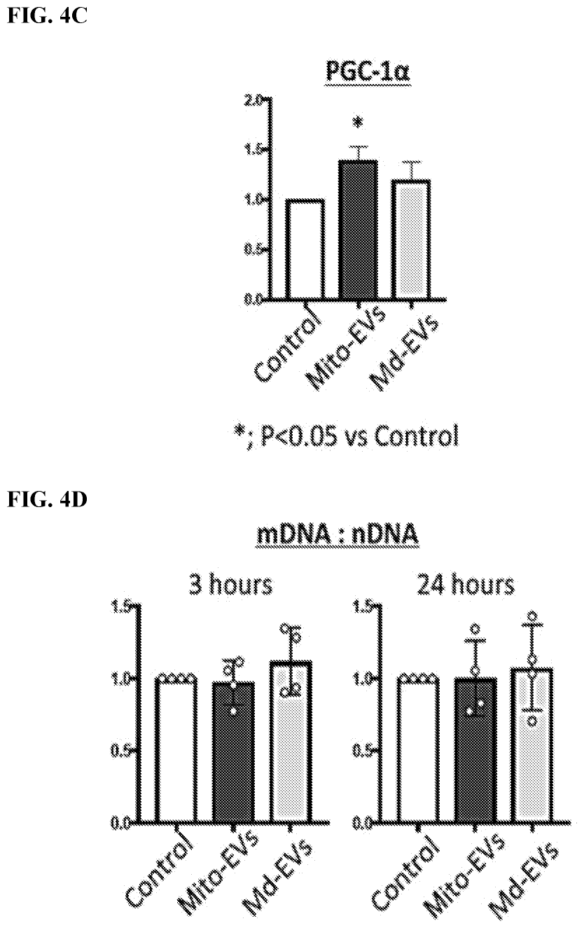

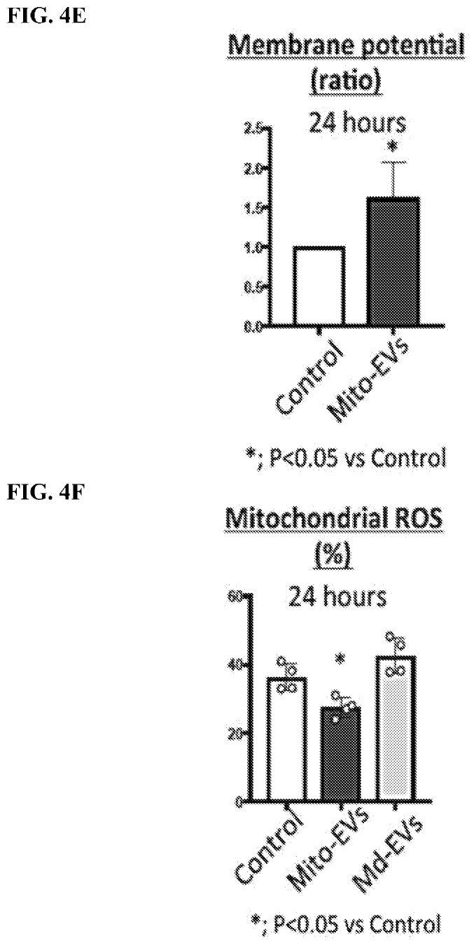

Human induced pluripotent stem cells (iPSCs) and iPSCs-derived cardiomyocytes (iCMs) were generated. See FIG. 1. Extracellular mitochondria were analyzed by flow cytometry (FCM), WB (Western blot) and TEM (transmission electron microscopy). See FIGS. 2A-2F. iCMs were exposed to hypoxia (1% 02, 24 hours). Cells were treated with 3.0×10⁸/mL. See FIGS. 3A-3D. Mito-EVs or isolated mitochondria that contained same amounts of mitochondria at the time of reoxygenation. ATP levels were measured at 3 and 24 hours after reoxygenation. See FIGS. 3A-3D. FCM (mitochondrial membrane potential and ROS) was performed at 6 hours after reoxygenation. WB of PGC-1α and contractility experiments were performed at 24 hours after reoxygenation. See FIGS. 4A-4F.

iCM were subjected to 24 hours hypoxia. Hypoxic iCM were treated with M-EVs at the time of reoxygenation. Treatment with M-EVs significantly increased intracellular ATP production restoring the viability of hypoxia injured iCMs (p<0.05, FIGS. 3A-3D). M-EVs increased functional mitochondria and reduced mitochondrial ROS in hypoxic iCM. M-EV treatment increased contractility and pGC-1α expression in hypoxic iCM.

### Example 3

### Intramyocardial Injection of Mitochondria-Containing Extracellular Vesicles Improved HF in Mice Model of MI

Mitochondrial dysfunction disrupts the balance between energy supply and demand in the failing heart. A novel therapy that promotes cellular energetics by providing autologous mitochondria enhances LVEF and prevents cardiac remodeling after myocardial injury is described herein.

It was hypothesized that mitochondria-containing extracellular vesicles restore mitochondrial function of the injured human cardiomyocytes.

Methods and Results

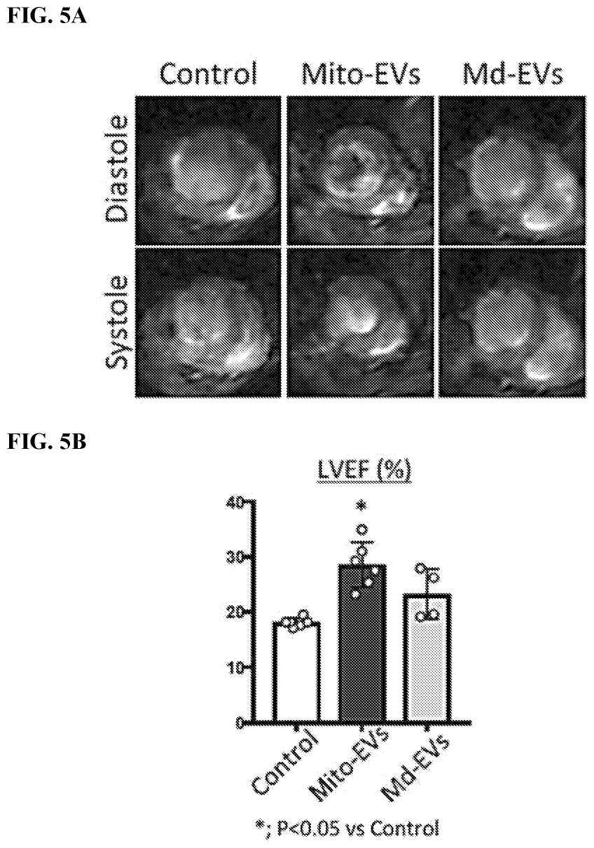

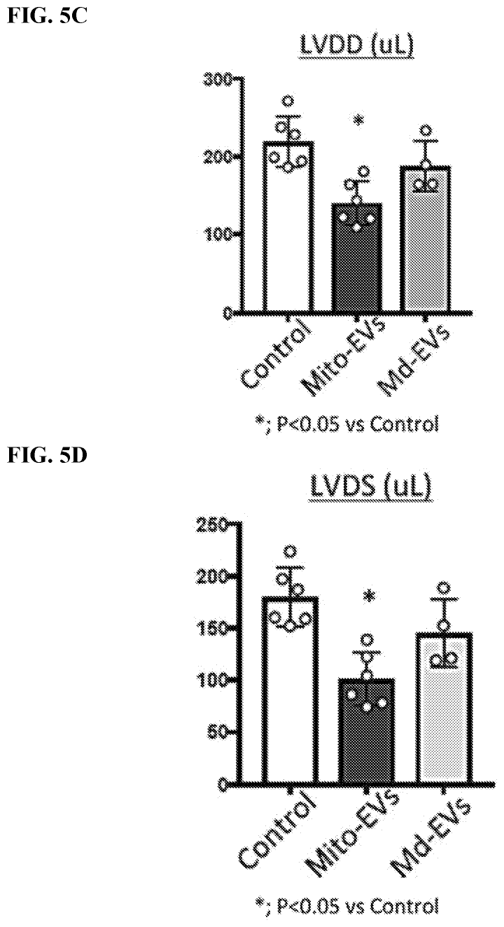

In a mouse myocardial infarction (MI) model, Mito-EVs (3.0×10⁸) with or without 200 nm filtration and isolated mitochondria that contained same amount of mitochondrial protein were injected into peri-infarct area. MRI was performed 2 and 4 weeks after MI. See FIGS. 5A-5D.

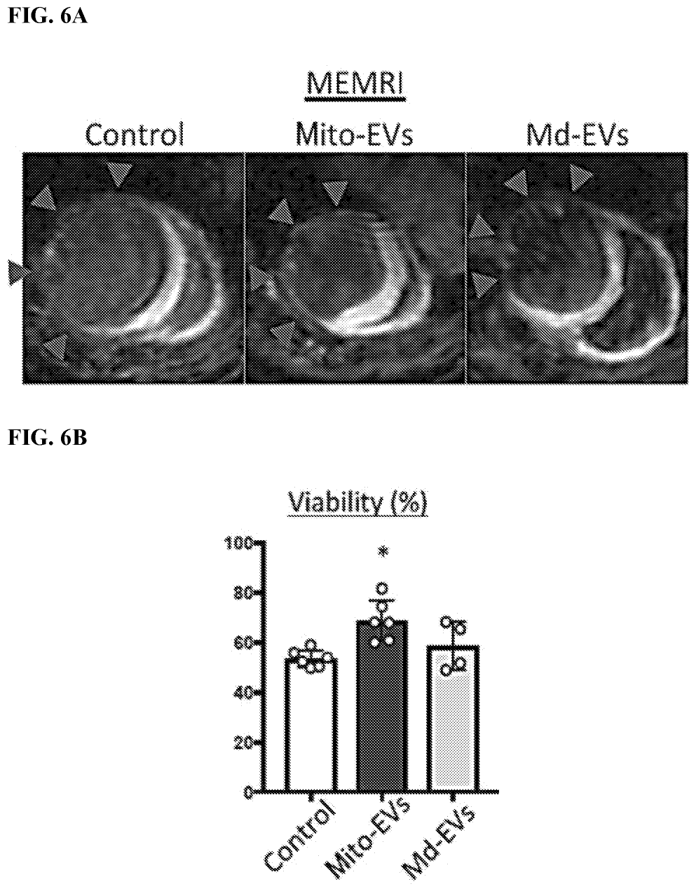

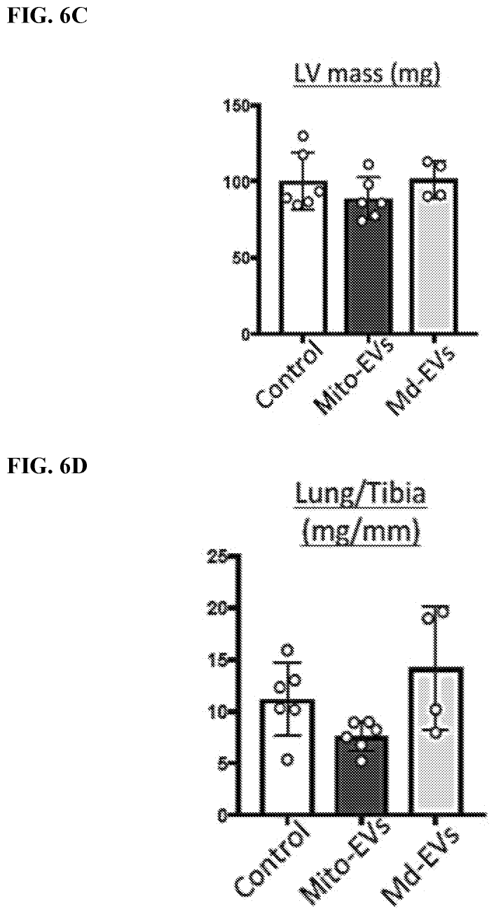

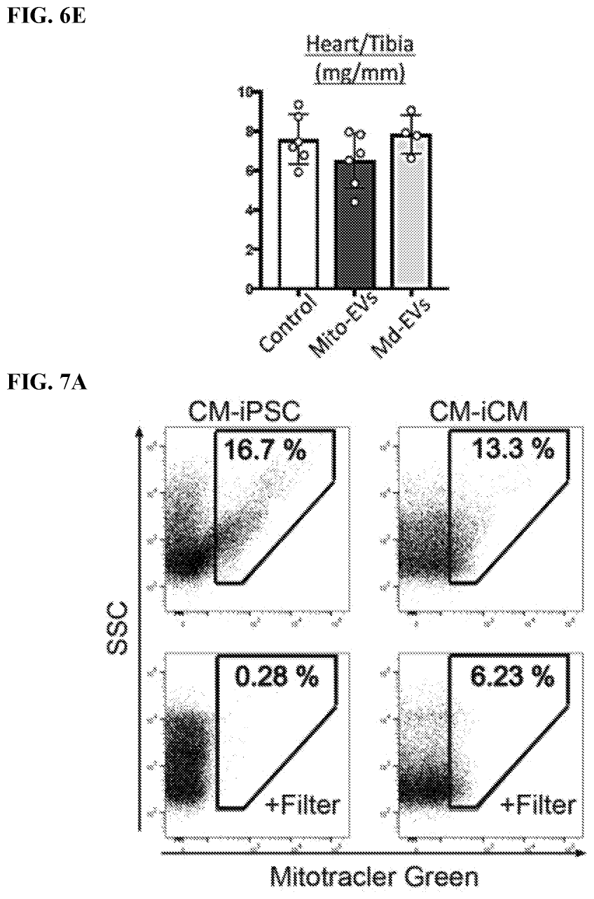

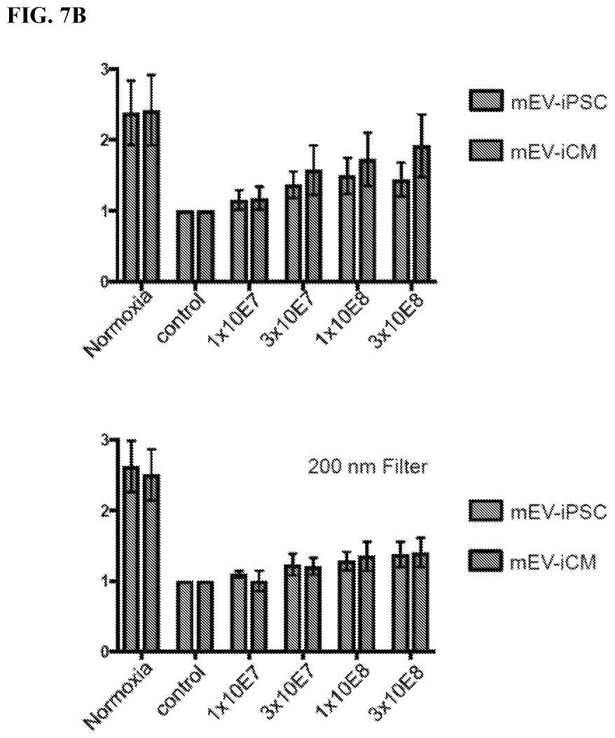

In an in-vivo mouse model of myocardial injury, intra-myocardial injection of M-EVs (3.0×10⁸) significantly improved left ventricular ejection fraction (LVEF) when compared to the same amount of isolated mitochondria (30.6%, vs. 19.3% at week 2, P<0.05, and 28.5% vs. 18.1% at week 4, P<0.05). Furthermore, MRI measurements, including left ventricular end-diastolic volume (LVEDV) and left ventricular endsystolic volume (LVESV) demonstrated significantly reduced left ventricular remodeling in MEV treated animals. Manganese-enhanced MRI (MEMRI) detected significantly higher percentage viable myocardium in the M-EV vs. isolated mitochondria treatment arm (68.8% vs. 53.4 at week 2 and 69.3% vs. 52.0 at week 4, P<0.05). At week 4, the measured heart weight (HW) and lung weight (LW), showed that the treatment with M-EV significantly reduced both HW and LW corrected by tibia length. These data suggested that M-EVs reduced the post-MI LV remodeling. See FIGS. 6A-6E.

This study identified M-EVs as an effective therapeutic for bioenergetics imbalance in HF. Proof-of-concept of M-EVs in a murine model of myocardial injury was demonstrated.

### Example 4

### Transfer of Mitochondria-Rich Extracellular Vesicles from iPSC-Derived Cardiomyocytes for Treatment of Heart Failure

Experiments have shown that extracellular vesicles efficiently transfer their cargo including microRNAs, small molecules, and proteins into the recipient cells, facilitating intercellular communications. Data shows that induced pluripotent stem cells (iPSCs)-derived cardiomyocytes (iCMs) produce larger-sized vesicles (200-500 nm) that contain functional mitochondria. These data suggest that the extracellular vesicles facilitate secure and highly efficient inter-cellular transfer of mitochondria. Data shows that the secretomes from immature iCMs contained mitochondria-positive extracellular vesicles (EVs) that improved mitochondrial function in the recipient cardiomyocytes. Mitochondria-containing EVs mediated transfer of mitochondria and immediately restored the bioenergetics of the recipient cardiomyocytes harboring dysfunctional mitochondria. Moreover, transfer of PGC-1α mRNA and proteins through the EVs sustained cellular bioenergetics even after the transferred mitochondria were degraded through mitochondrial autophagy. Finally, intra-myocardial injection of mitochondria-rich EVs enhanced survival of hypoxia-injured iCMs in vitro and improved ventricular function and attenuated cardiac remodeling of ischemia-injured murine myocardium in vivo.

Clinical application of intramyocardial injection of isolated-mitochondria into patients with HF is extremely challenging for several reasons; it is difficult for isolated mitochondria to (1) survive under extracellular environment with high calcium concentration, (2) be internalized by the recipient cardiomyocytes efficiently, and (3) produce enough ATP to improve intracellular bioenergetics in the cardiomyocytes. Here it has been demonstrated that EV-mediated transfer of mitochondria addresses these challenges. Data shown herein demonstrates that mitochondria inside EVs were protective, preventing calcium overload when compared to isolated-mitochondria. The lipid bilayer structure of the EVs prevented calcium ions from diffusing into the vesicles. EVs are internalized by recipient cells through rapid fusion with cell membrane, resulting in direct release of the mitochondrial content into the cytoplasm. Microscopic analysis revealed that the EVs facilitated quick transfer of EVs-mitochondria into recipient iCM. By contrast, isolated mitochondria were rarely internalized by recipient iCM within 24 hours. The data showed that the transferred mitochondria integrated with host mitochondria and increased ATP level within three hours. EV-mediated transfer of mitochondria solves poor efficiency of mitochondria transplantation using isolated-mitochondria.

### Example 5

### Microvesicles Rescue Patient-Specific iPSC-Derived Cardiomyocytes from Doxorubicin Injury

Anthracyclines are a highly effective class of chemotherapy used to treat common malignancies such as lymphoma, leukemia, and breast cancer. Anthracyclines carry a substantial lifetime risk of cardiomyopathy of 2-3% at commonly used doses. Anthracycline induced cardiomyopathy (AIC) carries a five-fold higher mortality than ischemic cardiomyopathy. Mesenchymal stem cell (MSC) therapy has demonstrated efficacy in animal models of AIC, and is now being studied in human trial by the Cardiovascular Cell Therapy Research Network in the SENECA trial (clinicaltrials.gov NCT02509156). Here, a clinical trial in a dish was executed by employing induced pluripotent stem cell derived cardiomyocytes (iCM) generated from trial patients to test the efficacy and mechanisms of the allogeneic MSCs used in the SENECA trial.

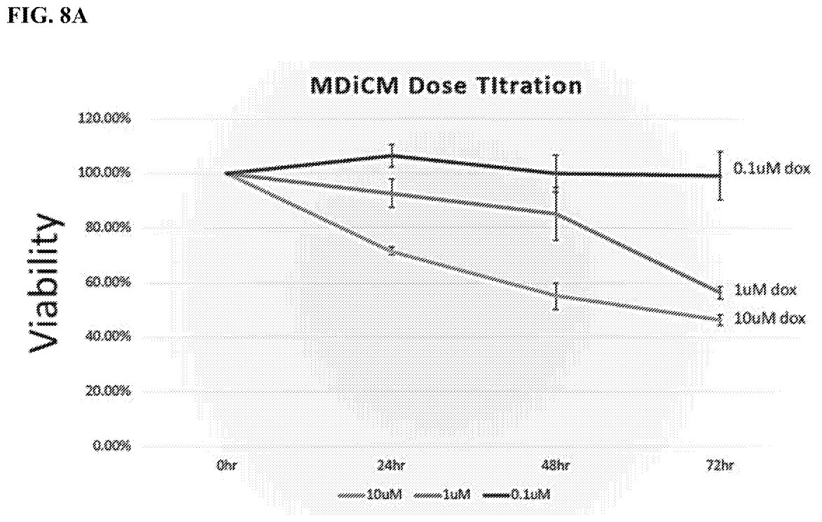

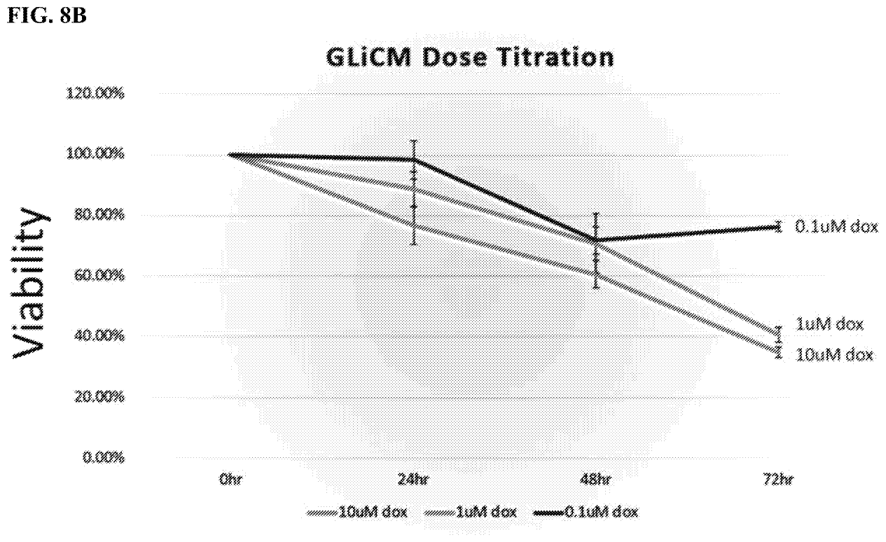

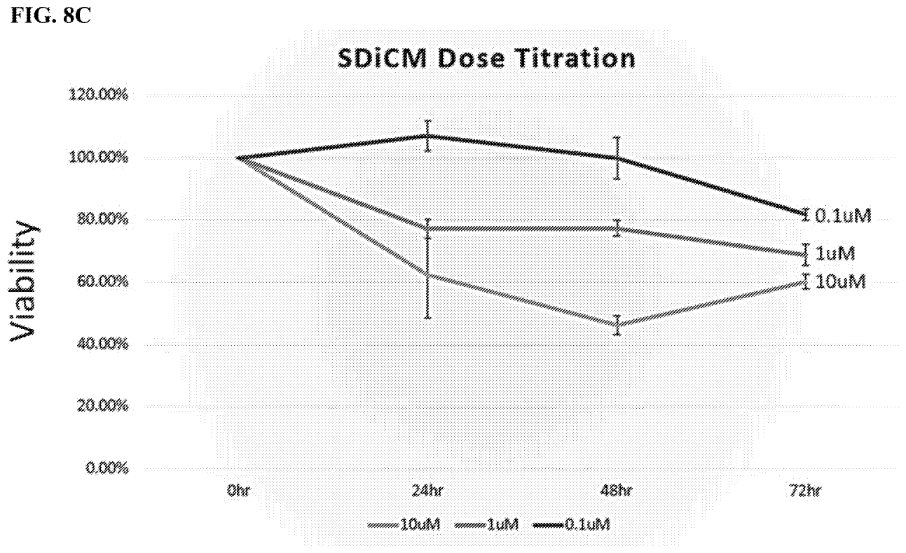

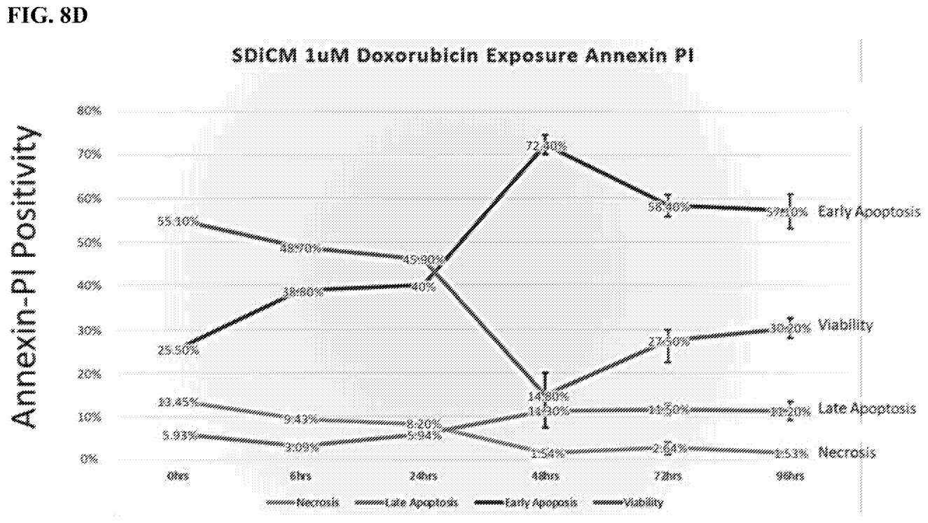

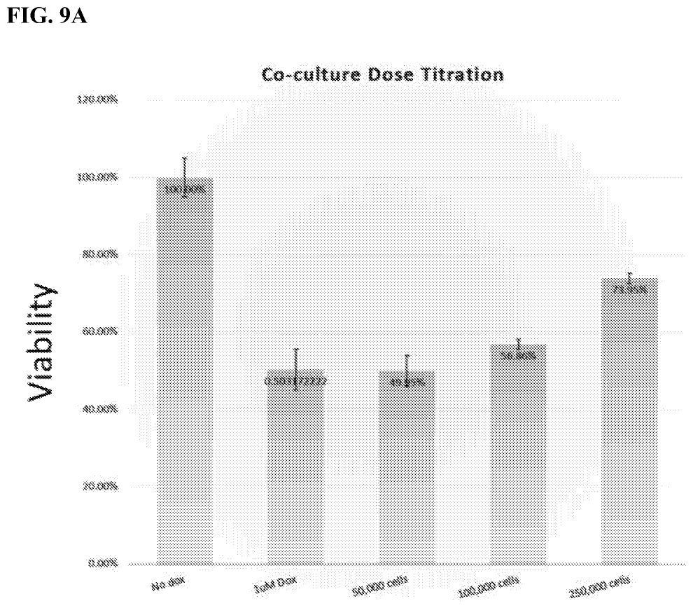

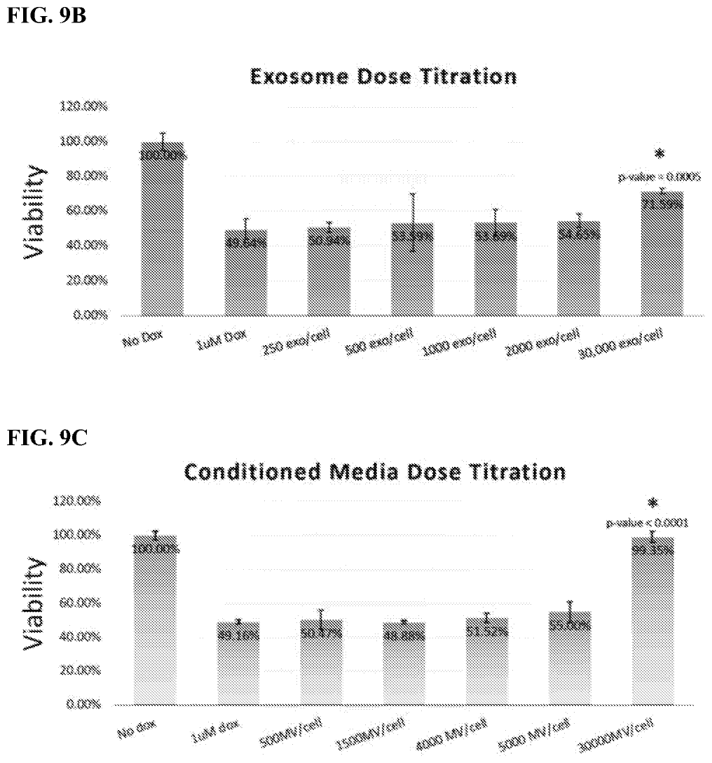

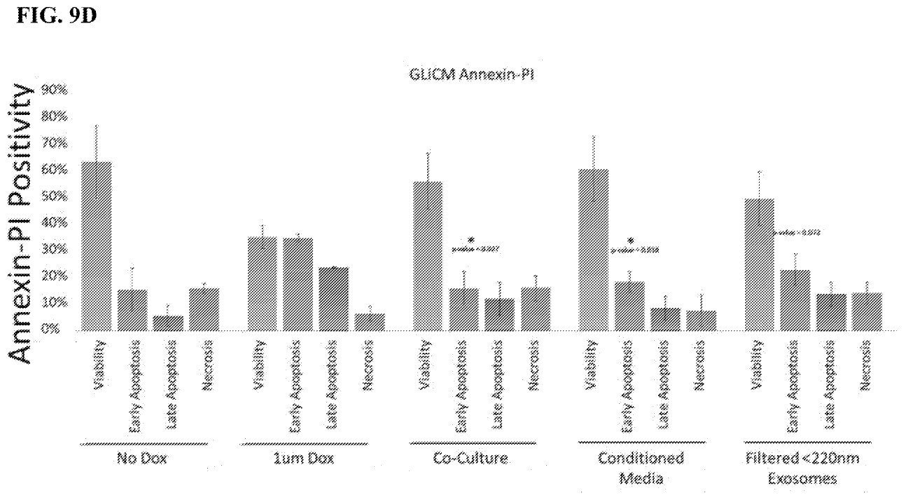

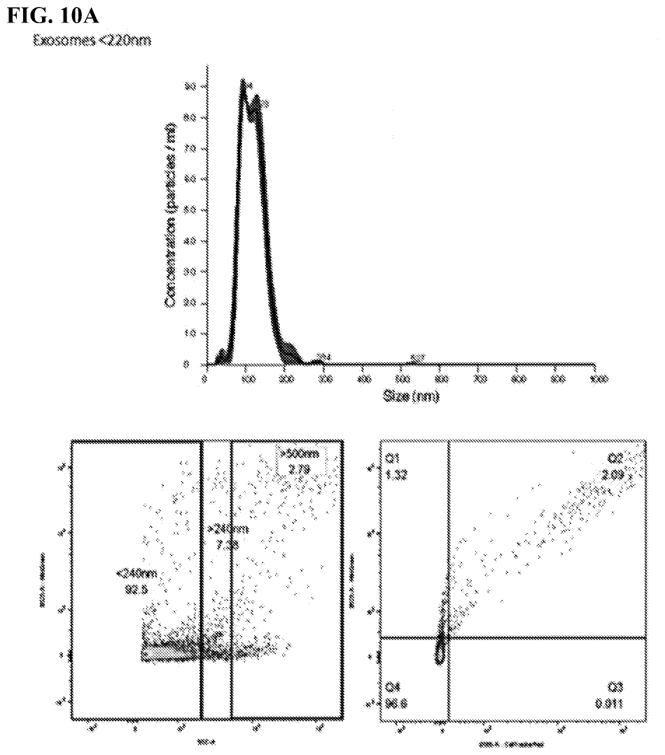

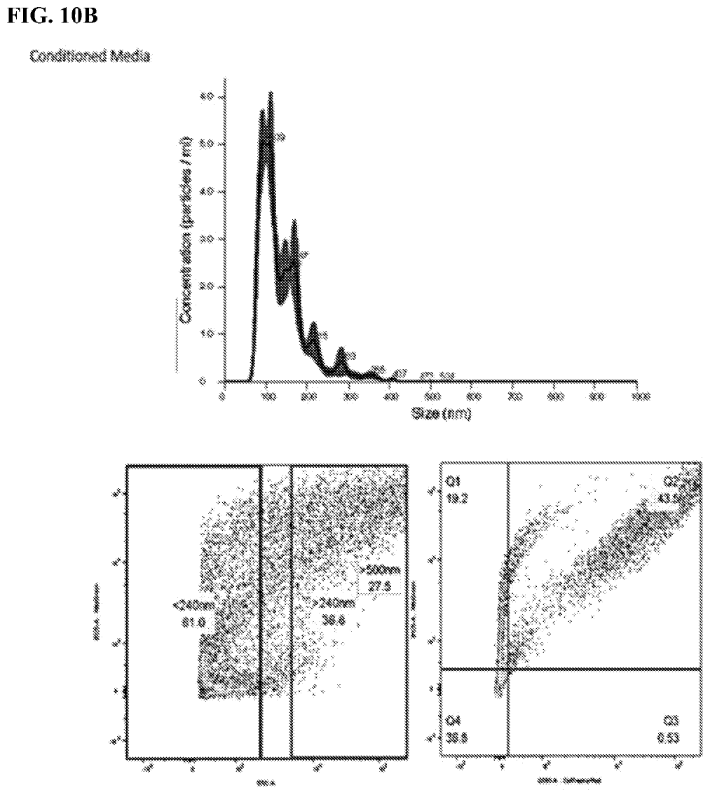

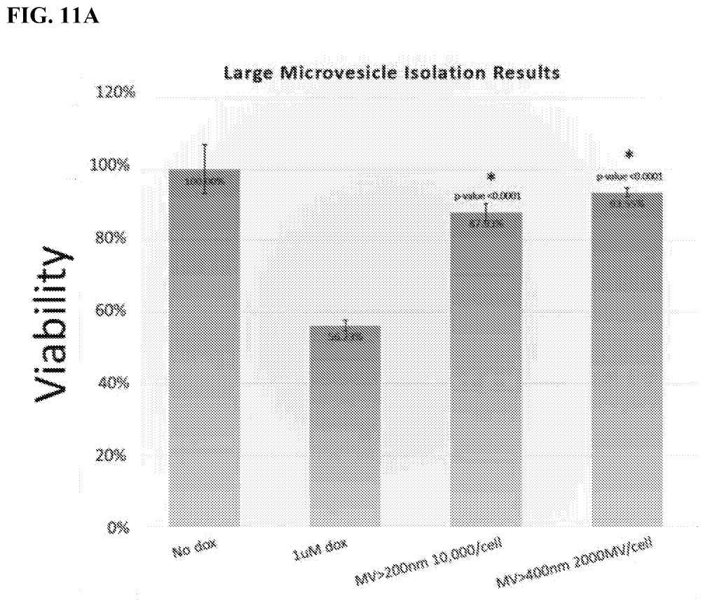

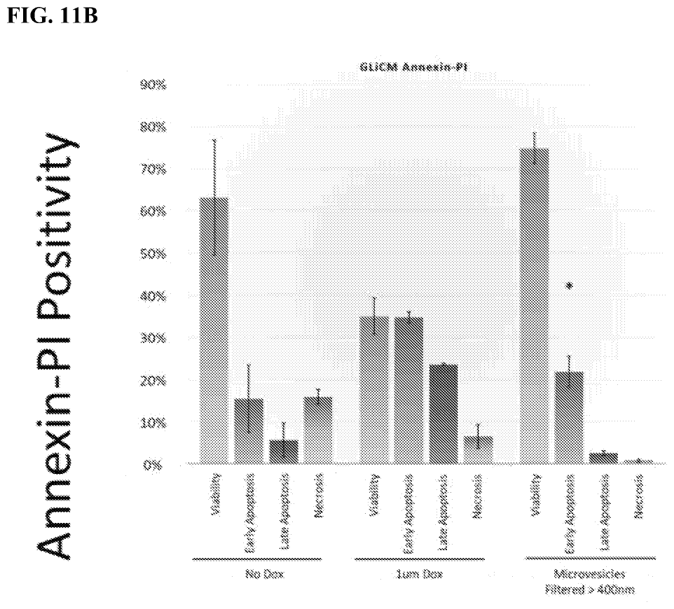

Mononuclear cells from three trial patients were successfully reprogrammed into induced pluripotent stem cells (iPSCs). All three were successfully differentiated into iCMs. MSC efficacy was first tested using a 1000 nm pore coculture system. MSCs were trypsinized and resuspended in a transwell insert and co-cultured with 2.5×10⁵ iCMs. On day zero, 1 μM doxorubicin was added to culture for 24 hrs. Media was exchanged on day one, and assays were performed on day two. To assess the therapeutic mechanisms by which MSCs affect iCM viability. Exosomes and microvesicles were independently tested. Exosomes were collected from MSC media by filtering with a 220 nm filter and incubating with PEG to facilitate exosome precipitation. Microvesicles were isolated using a novel filter system called EXOtic. See FIGS. 8A-8D, 9A-9D, 10A-10B, and 11A-11B.

Mesenchymal stem cells have the capacity to mitigate doxorubicin injury in patient-specific cardiomyocytes. This effect is likely mediated through microvesicles, and possibly due direct mitochondrial transfer. Further study into the role of mitochondria in this process as well as additional contents of microvesicles is needed. Using iCMs, performing a clinical trial in a dish is possible and may offer mechanistic insights into the results of the SENECA trial.

### P-Embodiments

Embodiment P-1. A method of treating a subject having a disorder characterized by intracellular bioenergetics imbalance in a cardiomyocyte, comprising administering a therapeutically effective amount of cardiomyocyte mitochondria-containing extracellular vesicles.

Embodiment P-2. A method of treating a subject having a disorder characterized by intracellular bioenergetics imbalance in a cardiomyocyte, comprising:
(i) obtaining a sample of blood;
(ii) isolating peripheral blood mononucleocyte cells (PBMCs) from the blood to obtain a PBMC fraction of PBMC's;
(iii) inducing pluripotency of said PBMC fraction of PBMC's thereby producing induced pluripotent stem (iPS) cells;
(iv) differentiating the iPS cells thereby producing cardiomyocytes;
(v) isolating cardiomyocyte mitochondria-containing extracellular vesicles produced from said cardiomyocytes; and
(vi) administering a therapeutically effective amount of said cardiomyocyte mitochondria-containing extracellular vesicles to the subject.

Embodiment P-3. The method embodiment P-1 or P-2, wherein the disorder is cardiomyopathy.

Embodiment P-4. The method of any one of the preceding Embodiments, wherein the cardiomyopathy is selected from hypertrophic, arrthymogenic right ventricular, dilated, restrictive, and ischemic cardiomyopathy.

Embodiment P-5. The method of any one of the preceding Embodiments, wherein the disorder is heart failure.

Embodiment P-6. The method of any one of the preceding Embodiments, wherein the extracellular vesicles are greater than 200 nm.

Embodiment P-7. The method of any of the preceding Embodiments, wherein the cardiomyocyte mitochondria-containing extracellular vesicles are autologous to the subject.

Embodiment P-8. A method for improving cellular function cardiomyocyte comprising contacting said cardiomyocyte with cardiomyocyte mitochondria-containing extracellular vesicles.

Embodiment P-9. The method of Embodiment P-8, wherein the extracellular vesicles are greater than 200 nm.

Embodiment P-10. The method of any one of Embodiment P-8 or P-9, wherein the cardiomyocyte is an injured cell.

Embodiment P-11. The method of Embodiment P-10, wherein the injured cell is an ischemic cell or a cell exposed to one or more chemotherapeutic agents.

Embodiment P-12. A method for improving viability of a cardiomyocyte comprising contacting said cardiomyocyte with a cardiomyocyte mitochondria-containing extracellular vesicles.

Embodiment P-13. The method of Embodiment P-12, wherein the extracellular vesicles are greater than 200 nm

Embodiment P-14. The method of Embodiment P-12 or P-13, wherein the cardiomyocyte is an injured cell.

Embodiment P-15. The method of Embodiment P-14, wherein the injured cell is an ischemic cell or a cell exposed to one or more chemotherapeutic agents.

Embodiment P-16. A method for improving the bioenergetics balance of a cardiomyocyte comprising contacting said cardiomyocyte with cardiomyocyte mitochondria-containing extracellular vesicles.

Embodiment P-17. The method of Embodiment P-16, wherein the extracellular vesicles are greater than 200 nm

Embodiment P-18. The method of Embodiment P-16 or P-17, wherein the cardiomyocyte is an injured cell.

Embodiment P-19. The method of Embodiment P-18, wherein the injured cell is an ischemic cell or a cell exposed to one or more chemotherapeutic agents.

Embodiment P-20. A composition comprising iPSC-derived cardiomyocytes, cell culture media, and extracellular vesicles, wherein the extracellular vesicles are greater than 200 nm and comprise mitochondria and/or mitochondrial fragments.

1. A method of treating a subject having a disorder characterized by intracellular bioenergetics imbalance in a cardiomyocyte, comprising administering a therapeutically effective amount of cardiomyocyte mitochondria-containing extracellular vesicles.

2. The method of claim 1 further comprising:
(i) obtaining a sample of blood;
(ii) isolating peripheral blood mononucleocyte cells (PBMCs) from the blood to obtain a PBMC fraction of PBMC's;
(iii) inducing pluripotency of said PBMC fraction of PBMC's thereby producing induced pluripotent stem (iPS) cells;
(iv) differentiating the iPS cells thereby producing cardiomyocytes; and
(v) isolating cardiomyocyte mitochondria-containing extracellular vesicles produced from said cardiomyocytes.

3. The method of claim 1, wherein the disorder is cardiomyopathy.

4. The method of claim 1, wherein the cardiomyopathy is selected from hypertrophic, arrthymogenic right ventricular, dilated, restrictive, and ischemic cardiomyopathy.

5. The method of claim 1, wherein the disorder is heart failure.

6. The method of claim 1, wherein the extracellular vesicles are greater than 200 nm.

7. The method of claim 1 wherein the cardiomyocyte mitochondria-containing extracellular vesicles are autologous to the subject.

8. A method for improving cellular function, viability, or bioenergetics balance of a cardiomyocyte comprising contacting said cardiomyocyte with cardiomyocyte mitochondria-containing extracellular vesicles.

9. The method of claim 8, wherein the extracellular vesicles are greater than 200 nm.

10. The method of claim 8, wherein the cardiomyocyte is an injured cell.

11. The method of claim 10, wherein the injured cell is an ischemic cell or a cell exposed to one or more chemotherapeutic agents.

12.-19. (canceled)

20. A composition comprising iPSC-derived cardiomyocytes, cell culture media, and extracellular vesicles, wherein the extracellular vesicles are greater than 200 nm and comprise mitochondria and/or mitochondrial fragments.
